(19) United States
(12) Patent Application Publication
(10) Pub. No.: US 2024/0315609 A1
(43) Pub. Date: Sep. 26, 2024

(54) ANALYTE DETECTION DEVICE WITH MULTILAYER ADHESIVE TAPE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/578,008

(22) PCT Filed: Dec. 9, 2021
(86) PCT No.: PCT/CN2021/136627

§ 371 Date: Jan. 10, 2024

(30) Foreign Application Priority Data
Aug. 6, 2021 (WO) PCT/CN2021/111219

(51) Int. Cl. A61B 5/145; A61B 5/00

(52) U.S. Cl. CPC A61B 5/14532; A61B 5/6833; A61B 2560/0443

(57) ABSTRACT

An analyte detection device with multilayer adhesive tape includes: an analyte detection module fixed on user's skin surface through an adhesive module, the adhesive module equipped with at least N (N≥2) layers of the adhesive tape. The first side of the first layer of the adhesive tape is fixedly connected with the analyte detection module, and the rest of the adhesive tape is stacked on the first side of the first layer of the adhesive tape. The working time of the adhesive module can be extended and the user experience can be improved through relay use of the inner and outer adhesive tape one by one, and the outer adhesive tape delays the edge warping of the inner adhesive tape.

### TECHNICAL FIELD

The invention mainly relates to the field of medical devices, in particular to an analyte detection device with multilayer adhesive tape.

### BACKGROUND

The pancreas in a normal person can automatically monitor the amount of glucose in the blood and automatically secrete the required dosage of insulin/glucagon. However, for diabetic patients, the function of the pancreas is abnormal, and the pancreas cannot normally secrete required dosage of insulin. Therefore, diabetes is a metabolic disease caused by abnormal pancreatic function and also a lifelong disease. At present, medical technology cannot cure diabetes, but can only control the onset and development of diabetes and its complications by stabilizing blood glucose.

Patients with diabetes need to check their blood glucose before injecting insulin into the body. At present, most of the detection methods can continuously detect blood glucose, and send the blood glucose data to the remote device in real time for the user to view. This detection method is called Continuous Glucose Monitoring (CGM), which requires the detection device to be attached to the surface of the patients' skin, and the sensor carried by the device is inserted into the subcutaneous tissue fluid for testing.

When the detection device is stuck to the user's skin surface, due to static electricity or skin movement and other reasons, in the process of use, as the use time increases, on the one hand, the sticky force of the adhesive tape becomes smaller, on the other hand, the outer edge of the tape warped, and the area of warped edge will increase, eventually leading to displacement or fall off of the detection device, affecting the normal use of the detection device.

Therefore, the existing technology is in urgent need of an analyte detection device that can prolong the working time of adhesive tape.

### BRIEF SUMMARY OF THE INVENTION

This invention implementation example discloses an analyte detection device with multilayer adhesive tape, the analyte detection module is fixed on the user's skin surface through the adhesive module, the adhesive module is equipped with at least N (N≥2) layers of adhesive tape, the first side of the first layer of adhesive tape is fixedly connected with the analyte detection module, and the rest of the adhesive tape is stacked on the first side of the first layer of adhesive tape, the working time of the adhesive module can be extended and user experience can be improved through the relay use of the inner and outer adhesive tape one by one, or the outer adhesive tape delay the edge warping of the inner adhesive tape.

The invention discloses an analyte detection device with multilayer adhesive tape, which comprises an analyte detection module for testing analyte parameter information; The adhesive module comprises N layers of adhesive tape. The first side of the first layer of adhesive tape is fixedly connected with the analyte detection module. The second side opposite to the first side of the first layer of adhesive tape is coated with adhesive material.

According to one aspect of the invention, the second layer to the Nth layer of adhesive tape are annular structure.

According to one aspect of the invention, the second layer to the Nth layer of adhesive tape are provided with at least one tearing part.

According to one aspect of the invention, the second layer to Nth layer of adhesive tape shall cover at least part of the adjacent inner layers.

According to one aspect of the invention, the material of adhesive tape is one of polyethylene, polypropylene, non-woven fabric or pure cotton.

According to one aspect of the invention, the thickness of the adhesive tape is 1˜500 um.

According to one aspect of the invention, the adhesive module also comprises at least one protective film.

According to one aspect of the invention, the protective film is located between adjacent adhesive tapes.

According to one aspect of the invention, the Rockwell hardness of protective film is higher than that of adhesive tape.

According to one aspect of the invention, the Rockwell hardness of the protective film is 80 to 100HRM.

According to one aspect of the invention, the thickness of the protective film is 10˜500 um.

According to one aspect of the invention, the outer edge of the protective film is suitable for the outer edge of the adjacent inner adhesive tape.

According to one aspect of the invention, the protective film is provided with at least one tearing part.

According to one aspect of the invention, the material of protective film is one of polycarbonate, polyamide, polyformaldehyde, polyphenylene ether, polyester, polyphenylene sulfide and poly-aryl ester.

According to one aspect of the invention, the analyte detection module comprises the transmitter unit, the sensor unit, the power supply and the bottom case. The sensor unit comprises the internal part and the external part.

According to one aspect of the invention, the first layer of adhesive tape is provided with the first through-hole, and the internal part passes through the first through-hole.

According to one aspect of the invention, at least one layer of release paper is provided on the second side of the first layer of adhesive tape before the adhesive module is pasted on the user's skin surface.

According to one aspect of the invention, the release paper is provided with a second through-hole, the position of the second through-hole corresponding to the first through-hole, so that the internal part of the sensor could pass through the second through-hole.

Compared with the prior art, the technical solution of the invention has the following advantages: The invention discloses an analyte detection device with multilayer adhesive tape, the analyte detection module is fixed on the user's skin surface through the adhesive module, the adhesive module comprises N layers of tape (N:2), the first side of the first layer of adhesive tape is fixedly connected with the analyte detection module, the second side opposite to the first side of the first layer of adhesive tape is coated with adhesive material, which is used for pasting the analyte detection module on the user's skin surface. The second layer to the Nth layer of adhesive tape are successively stacked on the first side of the first layer of adhesive tape, on the one hand, the outer layer of adhesive tape is successively stacked on the inner layer of adhesive tape, increasing the Hardness of the inner layer of adhesive tape, which can slow down the outer edge warping of the inner adhesive tape and prolong the working time of the adhesive module; On the other hand, if the outer layer of adhesive tape becomes warped or contaminated after using the detection device for a period of time, users can remove the outer layer of adhesive tape to leave a clean and complete inner layer of adhesive tape, which extends the working time of the adhesive module and improves the user experience.

Further, the second layer of adhesive tape to the Nth layer of adhesive tape is annular structure, when pasted on the first layer of adhesive tape, they can avoid interference with the analyte detection module, improve the appearance of adhesive tape and improve the user experience.

Further, in the adhesive tape from the second layer to the Nth layer, at least one layer of adhesive tape is provided with at least one tearing part, such as notch, perforated tear line, or broken seam, so as to tear off the outer layer of adhesive tape and reduce the impact on the inner layer of adhesive tape.

Further, the protective film is arranged between adjacent tapes to prevent adhesion between adjacent tapes and reduce the impact on the inner layer of adhesive tape when removing the outer layer of adhesive tape.

Further, the Rockwell hardness of the protective film is higher than that of the adhesive tape, and the contour of the outer edge of the tape is adapted to the contour of the outer edge of the tape, which can prevent the edge warping of the tape, prolong the service life of the tape, and improve the look and feel of the tape, and improve the user experience.

Further, the protective film is circular structure, which can avoid interference with the analyte detection module when pasting on the adhesive tape, and improve the look and feel of the adhesive tape and improve the user experience.

Further, the protective film is provided with at least one tearing part, such as notch, perforated tear line, or disconnect seam, etc., so as to reduce the influence on the inner layer of adhesive tape when the protective film is torn off.

Further, the second side of the first layer of adhesive tape is covered with at least one layer of release paper, which protects the adhesive material from contamination and prevents the adhesive tape from sticking to unrelated objects.

Further, the first through-hole is arranged on the first layer of adhesive tape, and the second through-hole is arranged on the release paper. The first through-hole corresponds to the position of the second through-hole, so that the internal part of the sensor can pass through the first through-hole and the second through-hole in turn.

### BRIEF DESCRIPTION OF THE DRAWINGS

FIG. 1a is a schematic diagram of the three-dimensional structure of an analyte detection device with multilayer adhesive tape according to an embodiment of the invention.

FIG. 1b is a schematic diagram of an analyte detection device with multilayer adhesive tape in another direction according to an embodiment of the invention;

FIG. 2 is a schematic diagram of analyte detection module structure according to an embodiment of the invention;

FIG. 3 is a schematic diagram of the adhesive module structure according to an embodiment of the invention;

FIG. 4a is a structural diagram of each layer of the adhesive module according to an embodiment of the invention;

FIG. 4b is a schematic diagram of the cascading sequence of the adhesive module according to an embodiment of the invention;

FIG. 4c is a structural diagram of each layer of the adhesive module according to another embodiment of the invention;

FIG. 4d is a schematic diagram of the cascading sequence of the adhesive module according to another embodiment of the invention;

FIG. 4e is a cascade schematic diagram of inner and outer layers of adhesive tape according to another embodiment of the invention;

FIG. 5 is a top view of the bottom case according to an embodiment of the invention;

FIG. 6a-FIG. 6b are schematic diagrams of the bottom board or the side wall of the bottom case before and after the failure according to an embodiment of the invention;

FIG. 6c-FIG. 6d are schematic diagrams of the second fastener part on the bottom case before and after the failure according to an embodiment of the invention.

### DETAILED DESCRIPTION

As mentioned above, when the prior art detection device is pasted onto the user's skin surface, Because of static electricity, skin movement, weak adhesive force of adhesive materials, etc., during use, the edge warping will appear on the outer edge of the adhesive tape on the detection device. With the increase of working time, the area of edge warping will increase, resulting in displacement or falling off of the detection device, affecting the normal use of the detection device and poor user experience.

In order to solve the problem, the invention provides an analyte detection device with multilayer adhesive tape, the analyte detection module is fixed on the user's skin surface through the adhesive module, and the adhesive module is provided with N (N≥2) layers of adhesive tape. The first side of the first layer of adhesive tape is fixedly connected with the analyte detection module, and the rest layer of the adhesive tape is stacked on the first side of the first layer of adhesive tape. Through the relay use of the inner and outer layer of adhesive tape, or the outer layer of adhesive tape delay the edge warping of the inner layer of adhesive tape, prolong the working time of the adhesive module, and improve the user experience.

Various exemplary embodiments of the invention will now be described in detail with reference to the drawings. The relative arrangement of the components and the steps, numerical expressions and numerical values set forth in the embodiments are not to be construed as limiting the scope of the invention.

In addition, it should be understood that, for ease of description, the dimensions of the various components shown in the figures are not necessarily drawn in the actual scale relationship, for example, the thickness, the width, the length or the distance of certain units may be exaggerated relative to other structures.

The following description of the exemplary embodiments is merely illustrative, and is not intended to be in any way limiting the invention and its application or use. The techniques, methods and devices that are known to those of ordinary skill in the art may not be discussed in detail, but such techniques, methods and devices should be considered as part of the specification.

It should be noted that similar reference numerals and letters indicate similar items in the following figures. Therefore, once an item is defined or illustrated in a drawing, it will not be discussed further in following description of the drawings.

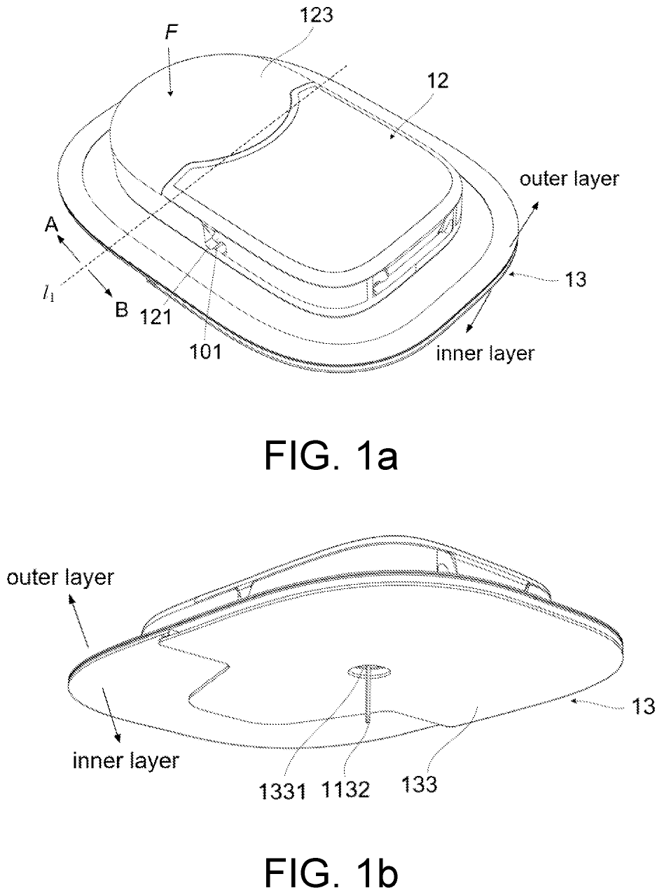

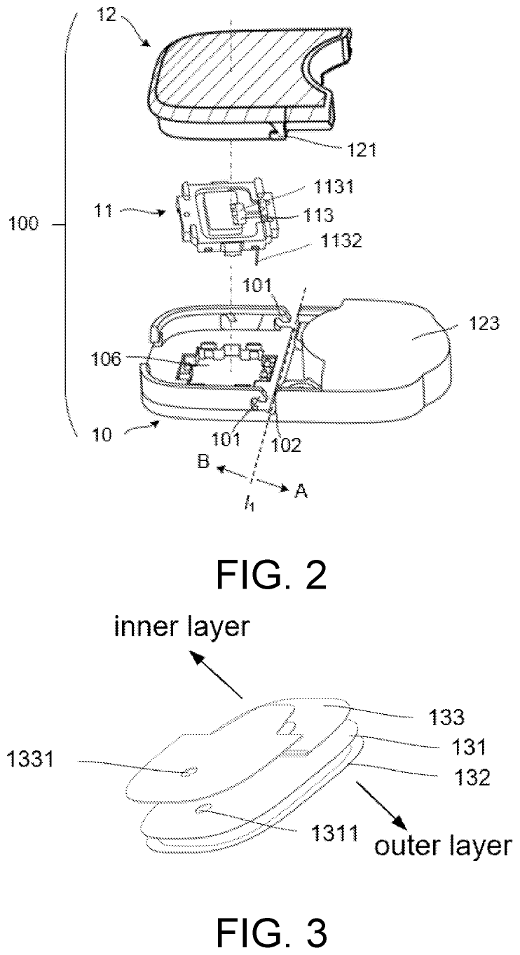

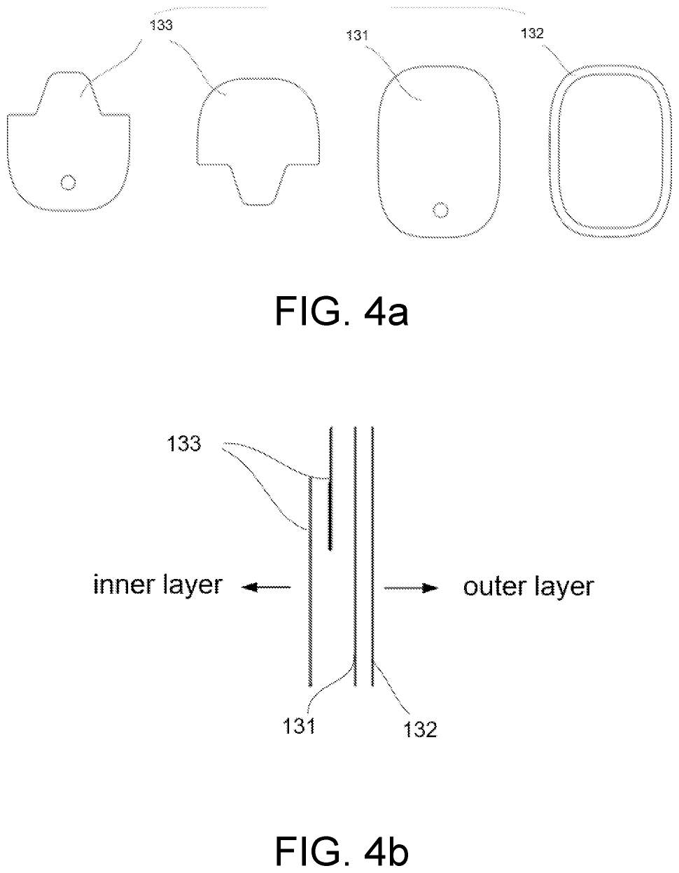

FIG. 1a is a schematic diagram of the three-dimensional structure of an analyte detection device with multilayer adhesive tape in an embodiment of the invention; FIG. 1b is a schematic diagram of an analyte detection device with multilayer adhesive tape in another direction in an embodiment of the invention. FIG. 2 is a schematic diagram of analyte detection module 100 in an embodiment of the invention. FIG. 3 is a structural diagram of the adhesive module 13 in an embodiment of the invention.

In the embodiment of the invention, the analyte detection device comprises analyte detection module 100 and adhesive module 13. Analyte detection module 100 comprises the bottom case 10, transmitter unit 12, sensor unit 11 and battery (not shown in the figure). The bottom case 10 is used to assemble transmitter unit 12 and sensor unit 11. Sensor unit 11 comprises external part 1131 and internal part 1132, the internal part 1132 is bent with respect to external part 1131.

In the embodiment of the invention, the adhesive module 13 comprises the first layer of adhesive tape 131, the first side (outer layer) of the first layer of adhesive tape 131 is fixedly connected with the bottom case 10, and the second side (inner layer) is coated with adhesive material for sticking the analyte detection device on the user's skin surface.

In the embodiment of the invention, the material of the first layer of adhesive tape 131 is one of polyethylene, polypropylene, non-woven fabric or pure cotton. The second side of the first layer of adhesive tape 131 is in direct contact with the user's skin. According to the actual use environment, the adverse reaction caused by the long-term contact of the first layer of adhesive tape 131 with the skin can be avoided by selecting the material mentioned above.

In order to adapt to the user's skin movement, such as zigzagging, stretching, etc., the thickness of the first layer of adhesive tape 131 is very thin, for example, about 1˜500 um. If the adhesive tape is too thin, there will be other problems, such as static electricity of the user's skin or violent skin movement, which will make the outer edge of the tape warped. Once the outer edge of the adhesive tape appears warped, the area of the warped edge of the first layer of adhesive tape 131 will gradually increase with the increase of use time, resulting in the reduction of the adhesive force between the first layer of adhesive tape 131 and the skin, which may lead to displacement or shedding of the analyte detection device. In addition, during use, the sticky force of the adhesive material decreases, resulting in displacement or shedding of analyte detection devices, which affects user experience.

In the embodiment of the invention, the protective film 132 is added to the outer edge of the first side of the first layer of adhesive tape 131. The Rockwell hardness of the protective film 132 is greater than that of the first layer of adhesive tape 131. In the preferred embodiment of the invention, the Rockwell hardness of the protective film 132 is 80˜100HRM.

In the preferred embodiment of the invention, the material of the protective film 132 is one of polycarbonate, polyamide, polyformaldehyde, polyphenylene ether, polyester, polyphenylene sulfide and poly-aryl ester.

In a preferred embodiment of the invention, the protective film 132 is poly-ethylene terephthalate (PET) with a Rockwell hardness of 90˜95HRM.

In the embodiment of the invention, the outer edge of the protective film 132 is fit with that of the first layer of adhesive tape 131. Here, “fit” means that the outer contour size, bending radius, shape and other parameters of the protective film 132 are consistent with the outer contour parameters of the first layer of adhesive tape 131, so that each of the outer edges of the first layer of adhesive tape 131 can fit the protective film 132.

In the embodiment of the invention, the thickness of the protective film is 10˜500 um.

In the embodiment of the invention, the protective film 132 is a annular structure, and the hollow structure of the annular structure can make the protective film stick to the outer layer of the first layer of adhesive tape 131 without interfering with the analyte detection module 100. Secondly, the inner edge of the protective film of the annular structure is consistent with the outer edge of the contour, which is more beautiful and enhances the user experience.

In the embodiment of the invention, the first through-hole 1311 is provided on the first layer of adhesive tape 131, and the position of the first through-hole 1311 corresponds to the internal part 1132 of the sensor, which is used to make the internal part 1132 of the sensor pass through to Pierce the user's skin.

In the embodiment of the invention, at least one layer of release paper 133 is provided on the inner surface of the first layer of adhesive tape 131, and the release paper 133 can prevent the adhesive material on the inner surface of the first layer of adhesive tape 131 from sticking and protect the adhesive material from contamination.

In the preferred embodiment of the invention, the release paper 133 is a single silicon release paper with a peeling force of 30˜50 g.

In the embodiment of the invention, the release paper 133 is provided with a second through-hole 1331, and the position of the second through-hole 1331 corresponds to the first through-hole 1311, so that internal part 1132 of the sensor can pass through the first through-hole 1311 and the second through-hole 1331 to Pierce the user's skin.

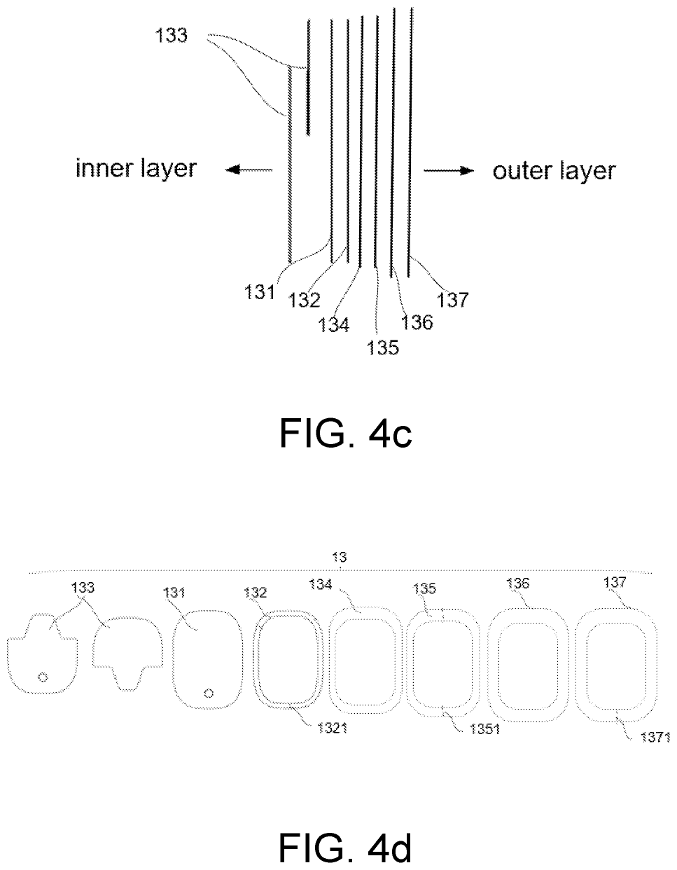

FIG. 4a is a structural diagram of each layer of the adhesive module 13 according to an embodiment of the invention; FIG. 4b is a schematic diagram of the overlapping sequence of the adhesive module 13 according to an embodiment of the invention; FIG. 4c is a structural diagram of each layer of the adhesive module 13 according to another embodiment of the invention; FIG. 4d is a schematic diagram of the overlapping sequence of adhesive module 13 according to another embodiment of the invention; FIG. 4e is a schematic diagram of laminated inner and outer layers of adhesive tape according to another embodiment of the invention.

In the embodiment of the invention, in order to facilitate the user to peel off the release paper 133 and the first layer of adhesive tape 131, the release paper 133 is preferably divided into two layers, with the peel ports facing inward to each other, and the peel ports of one layer bending outward and being covered by the peel ports of the other layer. In combination with the above mentioned, the adhesive module 13 consists of protective film 132, first layer tape 131 and release paper 133 from the outer layer to the inner layer.

In the embodiment of the invention, the second layer of adhesive tape 134 is arranged on the outer surface of the first layer of adhesive tape 131 in order to further prolong the working time of the adhesive module 13. The second layer of tape 134 is looped to avoid interference with analyte detection module 100.

In the embodiment of the invention, the outer edge of the second layer of adhesive tape 134 is fit with that of the first layer of adhesive tape 131. Here, “fit” means that the shape of the second layer of adhesive tape 134 is the same as that of the first layer of adhesive tape 131, but its contour size and bending radius can be changed according to actual needs. For example, in some embodiments, the outer edge of the second layer of adhesive tape 134 protrudes from the outer edge of the first layer of adhesive tape 131, after using the adhesive module 13 for a period of time, the outer edge of the second layer of adhesive tape 134 becomes warped, at this time, the second layer of adhesive tape 134 can be removed to expose the first layer of adhesive tape 131 without warping, and then the adhesive module 13 can be used normally, while the first layer of adhesive tape 131 is clean and tidy, maintaining the hygiene and aesthetic feeling of the adhesive module 13, and improving the user experience. For example, in some embodiments, the outer edge of the second layer of adhesive tape 134 overlaps with the outer edge of the first layer of adhesive tape 131, the second layer of adhesive tape 134 can strengthen the Rockwell hardness of the first layer of adhesive tape 131, and slow down the warping of the first layer of adhesive tape 131. For example, in some embodiments, the outer edge of the second layer of adhesive tape 134 is located inside the outer edge of the first layer of adhesive tape 131. When the outer edge of the first layer of adhesive tape 131 occurs warping, the second layer of adhesive tape 134 located inside can slow down the expansion of warping, so as to prolong the working time of the adhesive module 13. For example, one side of outer edge of the second layer of adhesive tape 134 protrudes from that of the first layer of adhesive tape 131, and the other side of the outer edge of is located inside that of the first layer of adhesive tape 131, forming the dislocation arrangement.

In the preferred embodiment of the invention, the protective film 132 is arranged between the second layer of adhesive tape 134 and the first layer of adhesive tape 131. Specifically, the protective film 132 is located on the contact surface of the second layer of adhesive tape 134 and the first layer of adhesive tape 131. The protective film 132 can reduce the effect on the first layer of adhesive tape 131 when the second layer of adhesive 134 is removed, and ensure that the first layer of adhesive tape 131 remains intact on the skin surface.

In another preferred embodiment of the invention, the tearing part 1321 is arranged on the protective film 132 for the convenience of tearing off the surface of the first layer of adhesive tape 131. The users can break off or tear off the protective film 132 at the tearing part 1321, and the effect on the inner adhesive tape can be reduced when the protective film 132 is torn off.

In the embodiment of the invention, the number of tearing parts 1321 can be one or multiple.

In an embodiment of the invention, the tearing part 1321 may be a notch, a perforated tear line or a slit, etc. Technical personnel in this field can understand that the specific structure of tearing part 1321 is not limited in the invention, as long as it is convenient for users to tear or break the protective film 132.

In the embodiment of the invention, the outer surface of the second layer of adhesive tape 134 can also be provided with the third layer of adhesive tape 136, and the utility of the third layer of adhesive tape 136 for the second layer of adhesive tape 134 is the same as that of the second layer of adhesive tape 134 for the first layer of adhesive tape 131, which will not be described here.

In the embodiment of the invention, the second protective film 135 is also arranged between the second layer of adhesive tape 134 and third layers of adhesive tape 136. The utility of the second protective film 135 is the same as that of the protective film 132, which will not be described here.

In the embodiment of the invention, the adhesive tape of adhesive module 13 is not limited to three layers, but may be more layers. The second layer of adhesive tape to the Nth (N≥2) layer of adhesive tape is superimposed layer by layer on the outer surface of the first layer of adhesive tape 131. Except for the first layer of adhesive tape 131, the rest of the tape and protective film are annular structure to avoid interference with the analyte detection module 100.

In the embodiment of the invention, one or more layers of protective film is arranged between two adjacent layers of adhesive tape to reduce the influence on the inner layer of adhesive tape when the outer layer of adhesive tape is torn off.

In other embodiments of the invention, one or more layers of protective film is arranged every two or three layers of adhesive tape, and no special restriction is made herein.

In an embodiment of the invention, the position relation of the outer adhesive tape relative to the inner adhesive tape is consistent with that of the second layer of adhesive tape 134 relative to the first layer of adhesive tape 131, and the outer adhesive tape can either completely cover the adjacent inner adhesive tape or partially cover the adjacent inner adhesive tape.

In the embodiment of the invention, at least one tearing part can also be arranged on the second layer of adhesive tape 134 to Nth layer to facilitate the user to tear off the adhesive tape. The structure of the tearing part is described above and will not be repeated here.

Continue with FIG. 1a, FIG. 1b and FIG. 2. Bottom case 10 comprises a fixed part and a force applying part. The bottom case 10 is provided with at least one second clamping part 101. The second clamping part 101 is used to clamping transmitter unit 12. Specifically, in an embodiment of the invention, the number of the second clamping part 101 is two. The two second clamping parts 101 correspond to the side walls of the bottom case 10.

Here, the fixed part and the force applying part are relative concepts. Depending on the structural design of the bottom case 10 and the transmitter unit 12, the location of the fixed part and the force applying part can be selected differently, as described in detail below.

Transmitter unit 12 is used to receive detection signals generated by sensors and transmit them wirelessly to remote devices. Therefore, the transmitter unit 12 is connected to the sensor.

The transmitter unit 12 is provided with at least one first clamping part 121. The first clamping part 121 corresponds to the second clamping part 101. The transmitter unit 12 is mounted on the bottom case 10 by clamping the second clamping part 101 and the first clamping part 121 to each other. Obviously, in an embodiment of the invention, the transmitter unit 12 is provided with two first clamping parts 121, that is, there are two pairs of first clamping parts 121 and second clamping parts 101.

Here, the first clamping part 121 corresponds to the second clamping part 101, which means they have the same number and corresponding positions.

When separating the bottom case 10 from transmitter unit 12, the fixed part is secured by finger or other device, bottom case 10 will fail by applying force in one direction with another finger or other auxiliary device, and then the second clamping part 101 and the first clamping part 121 are separated from each other, thus separating the transmitter unit 12 from the bottom case 10. That is, when separating bottom case 10 and transmitter unit 12, the user can separate them by applying force on the force applying part in one direction with only one finger, which is convenient for user operation. After separation, the transmitter can be reused, reducing the cost to the user.

It should be noted here that “fail” is a common concept in the field of engineering materials. After failure, the material loses its original function and the failure part cannot be restored again. Since the second clamping part 101 is part of the bottom case 10, the failure of bottom case 10 may occur in one or more of the following ways: failure of bottom plate, failure of side wall, or failure of the second clamping part 101. Obviously, after the failure of the bottom case 10, the bottom case 10 loses the function of clamping the transmitter unit 12.

The way of fixing the fixed part comprises clamping, supporting and other ways. There are no specific restrictions here, as long as the conditions for fixing the fixed part can be met.

Specifically, in the embodiment of the invention, the connection/i of the two second clamping parts 101 divides the bottom case 10 into A side and B side. A side is provided with a force applying part, B side is provided with a fixed part.

Case, the implementation of the invention, therefore, the separation of bottom case 10 and transmitter unit 12 process is as follows: Fix the fixed part on side B with one finger, and apply force F on the force applying part in one direction with the other finger, and then the second clamping part 101 fails, and the second clamping part 101 and the first clamping part 121 are separated, so that the transmitter unit 12 is separated from the bottom case 10.

It should be noted that the embodiment of the invention does not limit the position of the second clamping part 101. For example, the two second clamping parts 101 can be arranged on the bottom plate of the bottom case 10, and there is no specific restriction here.

The embodiment of the invention has no specific restriction on the shape of the top view of the detection device, and the shape can also be rounded rectangle, rectangle, circle, oval or other shapes.

The detection device of the embodiment of the invention also comprises a battery (not shown). The battery is used to supply power to the transmitter and is arranged in the bottom case 10. Battery part 123 is set in bottom case 10. Preferably, in embodiments of the invention, the top of the battery part 123 is flush with the top of the transmitter unit 12, thus reducing the thickness size of the detection device.

The battery part 123 can be directly used as the force applying part. Therefore, the battery part 123 is arranged on the A side of l₁. As the battery part 123 is thicker and has a relatively large area, it is easier for users to apply force to the battery part and optimize their operation steps.

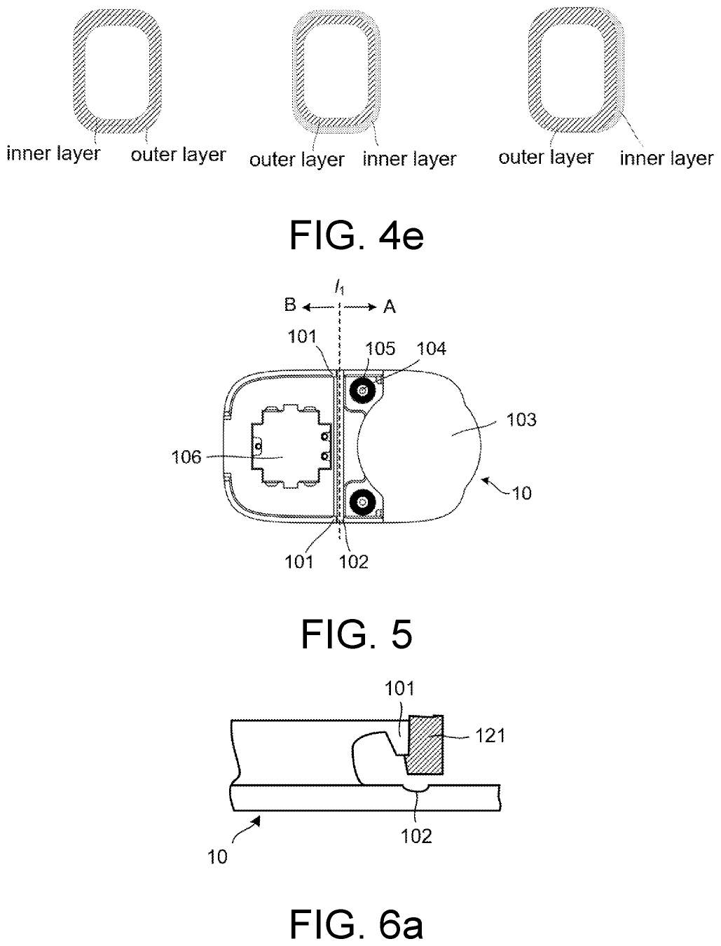

FIG. 5 is a top view of bottom case 10 in an embodiment of the invention.

As the battery needs to supply power to transmitter unit 12, the bottom case 10 is also provided with at least one connection hole 104 in an embodiment of the invention. Through connection hole 104, transmitter unit 12 is electrically connected to the positive and negative poles of the battery respectively.

An electrical contact that can be electrically connected to transmitter unit 12 can be set in connection hole 104. Or the transmitter unit 12 is provided with a protruding electrical connection end that can be inserted into a connection hole 104. Alternatively, the battery is electrically connected to the transmitter unit 12 by a wire passing through connection hole 104 or a conductive lead coated on the surface of the bottom case 10.

It should be noted that in other embodiments of the invention, no connection hole 104 can be set, and the transmitter unit 12 and the two poles of the battery can be electrically connected by a conductive lead completely coated on the surface of the bottom case 10. There is no specific restriction here.

Preferably, in an embodiment of the invention, a sealing ring 105 is arranged around the connecting hole 104 to seal the electrical connection position.

General, elastic sealing materials are pressed to seal. In the embodiment of the invention, the squeezed seal ring 105 exerts a certain force on transmitter unit 12. The sealing ring 105 provides the elasticity that facilitates the separation of the transmitter unit 12 from the bottom case 10 when force F is applied to the force applying part.

It should be noted that the sealing ring 105 or seals may not be set in other embodiments of the invention because of the good tightness of the transmitter unit 12 and the electric connection position of the battery or the transmitter unit 12 and the connection position of the sensor.

In the embodiment of the invention, a crease slot 102 is also provided at the position corresponding to the line l₁ on the bottom case 10. Crease slot 102 reduces the thickness of the bottom case, and the bottom case 10 is more likely to fail along the crease slot 102, making separation easier. Obviously, the crease slot 102 can run through the bottom plate of bottom case 10 along the line l₁.

Preferably, in an embodiment of the invention, the bottom plate of the bottom case 10 is also provided with mounting hole 106 and the sensor unit 11 arranged within the mounting hole 106. The outer contour of sensor unit 11 matches that of mounting hole 106. Here, the matching of the outer edges of the two means that the outer edges of the two can be interlaced.

The external part 1131 of sensor 113 is arranged on sensor unit 11, and the internal part 1132 is inserted subcutaneously. When installing the sensor 113, the sensor 113 is mounted to the bottom case 10 by the load of sensor unit 11. Preferably, in the embodiment of the invention, the shape of sensor 113 on sensor unit 11 does not change before and after installation, that is, the sensor 113 and sensor unit 11 are installed on the bottom case 10 at the same time. Therefore, after the installation of the sensor 113, sensor unit 11 becomes part of the bottom case 10.

It should be noted that in other embodiments of the invention, the detection device may be mounted directly to the skin surface as a whole without the use of sensor unit 11 to carry sensor 113, and no specific restrictions are hereby made.

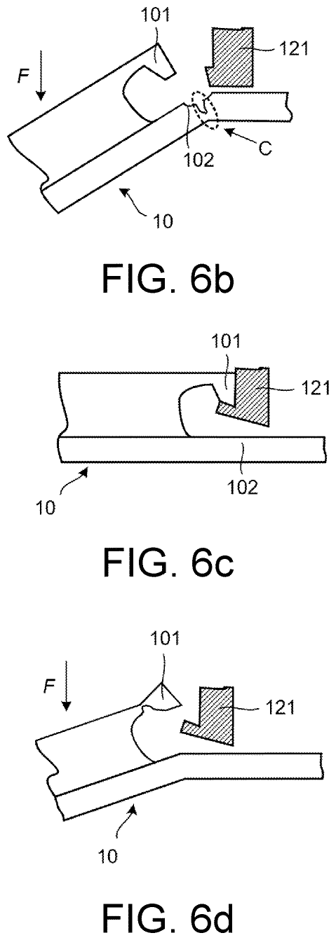

FIG. 6a and FIG. 6b are schematic diagrams of the side wall or bottom plate of bottom case 10 before and after failure, respectively. FIG. 6c and FIG. 6d are respectively schematic diagrams of the structure before and after the failure of the second clamping part 101 of bottom case 10.

As mentioned above, the first clamping part 121 and the second clamping part 101 are the clamping hooks. When the force F is applied to the force applying part in one direction, the bottom case 10 is broken along the crease slot 102, as shown by dotted ring C. In FIGS. 6c-6d, the second clamping part 101 is broken when force F is applied to the force applying part in one direction.

In other embodiments of the invention, the first clamping part 121 and the second clamping part 101 can be the clamping hole and the clamping block respectively, or the clamping block and the clamping slot respectively, and no specific restrictions are made here.

In other embodiments of the invention, the transmitter unit 12 may also be the force applying part, there is no specific limitation here. Here it should be pointed out that, although the force F may act directly on the transmitter unit 12, the actual effect of the force F is only shown on the bottom case 10, that is, the force F only change the morphology of bottom case 10 (such as the shape and structure change, etc.), didn't change the configuration of transmitter unit 12, at this time, it can still be considered that the user is the force applier to the force applying part of the bottom case 10.

To sum up, the invention discloses an analyte detection device with multilayer adhesive tape, the analyte detection module is fixed on the user's skin surface through the adhesive module, the adhesive module is equipped with at least N (N≥2) layers of adhesive tape, the first side of the first layer of adhesive tape is fixedly connected with the analyte detection module, and the rest of the adhesive tape is stacked on the first side of the first layer of adhesive tape, the working time of the adhesive module can be extended and user experience can be improved through the relay use of the inner and outer adhesive tape one by one, or the outer adhesive tape delay the edge warping of the inner adhesive tape.

While the invention has been described in detail with reference to the specific embodiments of the invention, it should be understood that it will be appreciated by those skilled in the art that the above embodiments may be modified without departing from the scope and spirit of the invention. The scope of the invention is defined by the appended claims.

1. An analyte detection device with multilayer adhesive tape, comprising:
an analyte detection module, configured to detect analyte parameter information;
an adhesive module configured to paste the analyte detection module on a surface, wherein the adhesive module comprises N layers of an adhesive tape, a first side of a first layer of the N layers of the adhesive tape is fixedly connected with the analyte detection module, a second side opposite to the first side of the first layer of the N layers of the adhesive tape is coated with adhesive material, a second layer to a Nth layer of the N layers of the adhesive tape are successively laminated and pasted on the first side of the first layer of the N layers of the adhesive tape, and N≥2.

2. According to the analyte detection device with multilayer adhesive tape mentioned in claim 1, wherein each of the second layer to the Nth layer of the N layers of the adhesive tape is an annular structure.

3. According to the analyte detection device with multilayer adhesive tape mentioned in claim 2, wherein at least a layer of the second layer to the Nth layer of the N layers of the adhesive tape is provided with at least one tearing part.

4. According to the analyte detection device with multilayer adhesive tape mentioned in claim 1, wherein each of the second layer to the Nth layer of the N layers of the adhesive tape covers at least part of an adjacent inner layer of the N layers of the adhesive tape.

5. According to the analyte detection device of multilayer adhesive tape mentioned in claim 1, wherein a material of the N layers of the adhesive tape is polyethylene, polypropylene, non-woven fabric or pure cotton.

6. According to the analyte detection device with multilayer adhesive tape mentioned in claim 1, wherein a thickness of the N layers of the adhesive tape is 1˜500 um.

7. According to the analyte detection device with multilayer adhesive tape mentioned in claim 1, wherein the adhesive module also comprises at least one protective film.

8. According to the analyte detection device with multilayer adhesive tape of claim 7, wherein the protective film is located between two adjacent layers of the N layers of the adhesive tape.

9. According to the analyte detection device with multilayer adhesive tape mentioned in claim 7, wherein a Rockwell hardness of the protective film is greater than a Rockwell hardness of the N layers of the adhesive tape.

10. According to the analyte detection device with multilayer adhesive tape described in claim 9, wherein the Rockwell hardness of the protective film is 80 to 100HRM.

11. According to the analyte detection device with multilayer adhesive tape mentioned in claim 7, wherein a thickness of the protective film is 10˜500 um.

12. According to the analyte detection device with multilayer adhesive tape mentioned in claim 7, wherein an outer edge contour of the protective film is fit with an outer edge contour of an inner layer of the two adjacent layers.

13. According to the analyte detection device with multilayer adhesive tape of claim 7, wherein the protective film is provided with at least one tearing part.

14. According to the analyte detection device with multilayer adhesive tape mentioned in claim 7, wherein a material of the protective film is one of polycarbonate, polyamide, polyformaldehyde, poly-phenyl ether, polyester, poly-phenyl sulfide and poly-aryl ester.

15. According to the analyte detection device with multilayer adhesive tape mentioned in claim 1, wherein the analyte detection module comprises a transmitter unit, a sensor unit, a battery and a bottom case, and the sensor unit comprises an internal part and an external part.

16. According to the analyte detection device with multilayer adhesive tape mentioned in claim 15, wherein the first layer of the N layers of the adhesive tape is provided with a first through-hole, and the internal part passes through the first through-hole.

17. According to the analyte detection device with multilayer adhesive tape mentioned in claim 16, wherein at least one layer of a release paper is provided on the second side of the first layer of the N layers of the adhesive tape before the adhesive module is pasted on the surface.

18. According to the analyte detection device with multilayer adhesive tape mentioned in claim 17, wherein a second through-hole is arranged on the at least one layer of the release paper, and a position of the second through-hole corresponds to the first through-hole, so that the internal part of the sensor unit passes through the second through-hole.
